(19) United States
(12) Patent Application Publication
(10) Pub. No.: US 2019/0175611 A1
(43) Pub. Date: Jun. 13, 2019

(54) Remyelination Therapy

(71) Applicant: The Regents of the University of California, Oakland, CA (US)

(72) Inventors: Riley Bove, Berkeley, CA (US); Jonah Chan, Burlingame, CA (US); Ari Green, San Anselmo, CA (US); Yun-An Shen, San Francisco, CA (US); Kelsey Rankin, San Francisco, CA (US)

(73) Assignee: The Regents of the University of California, Oakland, CA (US)

(21) Appl. No.: 16/324,662

(22) PCT Filed: Aug. 11, 2017
(86) PCT No.: PCT/US2017/046624

§ 371 Date: Feb. 11, 2019

Related U.S. Application Data
(60) Provisional application No. 62/374,270, filed on Aug. 12, 2016.
(60) Provisional application No. 62/430,357, filed on Dec. 6, 2016.

(51) Int. Cl. A61K 31/55; A61K 31/565

(52) U.S. Cl. CPC A61K 31/55; A61K 31/565

(57) ABSTRACT

The invention comprises the administration of a remyelinating agent to treat a demyelinating condition, such as MS. Disclosed are various remyelinating compositions which promote remyelination, oligodendrocyte differentiation, and the treatment of demyelinating conditions such as MS. In one aspect, the remyelinating compositions are selective estrogen receptor modulators with remyelinating properties. In one aspect, the remyelinating compositions are agonists of GPR56, a G-protein coupled receptor which has not previously been identified as an effector of remyelination.

### CROSS-REFERENCE TO RELATED APPLICATIONS

This application claims the benefit of priority to U.S. Provisional Application Ser. No. 62/374,270 entitled “Remyelination Therapy,” filed Aug. 12, 2016, and U.S. Provisional Application Ser. No. 62/430,357 entitled “Remyelination Therapy,” filed Dec. 6, 2016, the contents of each being hereby incorporated by reference.

### BACKGROUND OF THE INVENTION

In various demyelinating diseases, such as Multiple sclerosis (MS) the myelin coating that surrounds nerve fibers is attacked and damaged by the immune system or other factors. Myelin repair, or remyelination, is carried out by myelin-producing oligodendrocytes. In healthy animals, following demyelination processes, oligodendrocyte progenitor cells (OPCs) are activated and mature into myelin-producing oligodendrocytes, which wrap the axons and reapply myelin to damaged areas.

However, in various demyelinating diseases such as MS, the remyelination process is deficient and myelin damage accumulates over time, leading to severe degeneration. It is generally accepted that remyelination failure is not a result of impaired OPC recruitment and/or migration to demyelinated tissues. Instead, it is believed that a failure of OPC differentiation and membrane wrapping is the critical barrier impeding myelin repair in demyelinating conditions.

Current MS treatments address the underlying inflammatory component of the disease, but to date, there are no approved therapies for axon repair or remyelination. Accordingly, there is an ongoing and significant need in the art for effective remyelination therapies.

### SUMMARY OF THE INVENTION

The inventors of the present disclosure have advantageously developed novel therapies that promote remyelination. In a first aspect, the inventors of the present disclosure have identified certain selective estrogen receptor modulators (SERMs) that promote remyelination. In another aspect, the inventors of the present disclosure have determined that GPR56, a G-coupled protein receptor active in certain cells, is a mediator of the remyelination process and that agonists of this receptor can promote remyelination and associated processes in demyelinated tissues.

Additionally, the inventors of the present disclosure have determined that administration of remyelinating SERMs or GPR56 agonists in combination with estrogenic substances further promotes remyelination.

In one aspect, the scope of the invention encompasses novel therapeutic compositions comprising remyelinating SERMs. In one aspect, the scope of the invention encompasses novel therapeutic compositions comprising GPR56 agonists. In one aspect, the scope of the invention encompasses novel therapeutic compositions comprising remyelinating SERMs which also act as GPR56 agonists

In another aspect, the scope of the invention encompasses novel methods of using remyelinating SERMs and/or GPR56 agonists for the treatment of demyelinating conditions, promoting remyelination, and promoting the differentiation of oligodendrocyte precursors.

### BRIEF DESCRIPTION OF THE DRAWINGS

FIG. 1. BIMA micropillar assay results showing the percentage of rings stained positive for PDGFRα (indicative of OPCs) or myelin basic protein (MBP) (indicative of myelinating oligodendrocytes) in response to treatment with various agents.

FIG. 2. BIMA micropillar assay results showing the percentage of rings stained positive for MBP for BZA-treated cells at different dosages, with and without estradiol.

FIG. 3. Adenomatous polyposis coli (APC-a maker of mature oligodendrocytes) positive cell density and Olig2 (a marker of oligodendroglial lineage) cell density in rat corpus callosum following lysolecithin-induced demyelination and subsequent treatment with BZA.

FIGS. 4A, 4B, 4C, and 4D. FIGS. 4A, 4B, 4C, and 4D depict the percentage of MBP-positive cells following BZA treatment of isolated OPCs grown in culture. FIG. 4A depicts the BZA response in wild-type cells, FIG. 4B depicts the BZA response in ERα knockout cells, FIG. 4C depicts the BZA response in ERβ knockout cells, and FIG. 4D depicts the BZA response in ERα/ERβ double knockout cells.

### DETAILED DESCRIPTION OF THE INVENTION

Demyelinating Diseases.

In one aspect, the various embodiments of the invention are directed to the treatment of a demyelinating condition. A demyelinating condition, as used herein, refers to any disease state, autoimmune disorder, or process wherein demyelination, i.e. damage to the protective myelin sheath that surrounds nerve fibers, is occurring. Typically, such damage is caused by autoimmune processes of unknown origin.

In one embodiment, the demyelinating condition is a condition of the central nervous system. In one embodiment, the demyelinating disease is a myelinoclastic disorder. In one embodiment, the demyelinating condition is MS. In other embodiments, the demyelinating disease may be Devic's disease, an inflammatory demyelinating disease, or an acute disseminated encephalomyelitis. The demyelinating condition may comprise a leukodystrophic disorder. The demyelinating condition may comprise a central nervous system neuropathy, central pontine myelinolysis, or progressive multifocal leukoencephalopathy. The compositions and methods of the invention may also be applied to promote remyelination in demyelinating conditions of the peripheral nervous system.

Remyelinating Compositions.

The inventors of the present disclosure have identified certain compositions of matter that can act as remyelinating agents. The scope of the invention encompasses the administration of a what will be referred to herein as a “remyelinating composition.” A remyelinating composition comprises one or more remyelinating agents.

In a first aspect, the remyelinating composition comprises a SERM with remyelinating activity. Such compositions will be referred to herein as “remyelinating SERMs.” SERMs are compositions known to act on estrogen receptors, with tissue-specific effects, for example, acting as estrogenic agents in some tissues while being anti-estrogenic in other tissues.

In a second aspect, the remyelinating composition comprises a GPR56 agonist. GPR56, as known in the art, is G-coupled protein receptor active in various tissues. The GPR56 protein sequence has accession number Q9Y653, and a GeneCard ID of GC16P057610 and an NCBI Reference Sequence of NG_011643.1. GPR56 is also known as Adhesion G Protein-Coupled Receptor G1, and as TM7XN1.

GPR56 has previously been implicated in brain cortical patterning during development, ovarian development, cell adhesion, cell-cell interactions, and the maintenance of hematopoietic stem cells and/or leukemia stem cells in bone marrow niche. No role for GPR56 in myelination has previously been reported.

The inventors of the present disclosure have advantageously determined that GPR56 is implicated in remyelination. Specifically, certain remyelinating compositions have been identified which comprise SERMs and which also act as GPR56 agonists. This discovery provides the art with various methods of treatment, secondary uses of known compounds, and other valuable inventions which may be applied to promote remyelination.

Various embodiments of the invention are directed to activation of GPR56 by a GPR56 agonist. An agonist, as used herein, refers to any composition of matter which binds to GPR56 and induces one or more GPR56-associated myelination processes in oligdendroglia or other cells. The GPR56 Agonist may be a small molecule, growth factor, polypeptide, nucleic acid, or any other chemical or biological composition of matter.

GPR56 agonists, as used herein, will further refer to agonists, activators, and enhancers of GPR56's downstream effectors, i.e., activators of species which are regulated by GPR56 activation and which contribute to the remyelination response induced by GPR56 activation.

The inventors of the present disclosure have advantageously identified several remyelinating compositions. The remyelinating composition comprise remyelinating SERMs and also comprise putative GPR56 agonists.

A first remyelinating composition is bazedoxifene. Bazedoxifene (BZA) is a selective estrogen receptor modulator (SERM) which, in combination with conjugated estrogens, has been approved for the treatment of postmenopausal osteoporosis and of menopausal hot flashes. BZA in combination with conjugated estrogens is well tolerated, for example, having lesser side effects than tamoxifen.

A second remyelinating composition is raloxifene. Raloxifene is an FDA-approved drug, used in the prevention of osteoporosis and as a breast cancer preventative in postmenopausal women.

A third remyelinating composition is clomifene. Clomifene is an FDA-approved drug, used in fertility treatment in women.

A fourth remyelinating composition is toremifene. Toremifene is an-FDA approved drug, used in the treatment of breast cancer.

A fifth remyelinating composition is lasofoxifene. Lasofoxifene an EU-approved drug, and has shown efficacy in treating osteoporosis, breast cancer prevention, and vaginal atrophy.

A sixth remyelinating composition is ospemifene. Ospemifene is FDA-approved for the treatment of dyspareunia.

A seventh remyelinating composition is diarylpropionitrile. Diarylpropionitrile is an estrogen receptor agonist and is an activator of the endogenous oxytocin system.

An eighth remyelinating composition agonist is tamoxifen. Tamoxifen is widely used in the treatment of certain breast cancers.

Interestingly, each of the afore-listed compounds is a SERM. However, the inventors of the present disclosure have determined that the remyelination effects of these SERMs is mediated independently of either the Alpha or Beta estrogen receptors. As described in the Examples, below, remyelination is induced by these compounds in oligodendroglia even in cells wherein both forms of the ER have been knocked out. Accordingly, the remyelination effects induced by these compounds are largely independent of ER activity and are believed to be largely or wholly by action of GPR56 activation.

The scope of the invention encompasses various remyelinating compositions compositions. In one embodiment, the scope of the invention encompasses a pharmaceutical composition comprising remyelinating composition for the treatment of a demyelinating condition. In one embodiment, the remyelinating composition is a remyelinating SERM. In one embodiment, the remyelinating composition is a GPR56 agonist. In one embodiment, the remyelinating composition is selected from the group consisting of bazedoxifene, raloxifene, clomifene, ospemifene, lasofoxifene, diarylpropionitrile, toremifene, and tamoxifen, which are remyelinating SERMs and act as GPR56 agonists.

Co-Administration with Estrogens.

The inventors of the present disclosure have determined that the remyelinating effects of the remyelinating compositions are enhanced when co-administered with estrogenic substances. It is known in the art that MS has an estrogenic component, based on various associations such as an increased incidence of MS in women vs. males and decreased incidence of MS onset in post-menopausal women. However, the role of estrogenic hormones in MS is not clear, and administration of estrogenic hormones alone has not been shown to effectively treat demyelinating conditions. Accordingly, the surprising enhancement of remyelination by estrogenic hormones in combination with remyelinating compositions provides the art with a remyelination therapy of increased potency.

The co-administered estrogenic agent may comprise any estrogen hormone or estrogenic substance, for example, such as estrogen, estradiol, conjugated estrogens, estriol, estrogen mimics, estrone, ethynil estrogen, and estrogen agonists.

Methods of the Invention.

The scope of the invention encompasses various methods of utilizing remyelinating compositions to promote remyelination processes. The methods disclosed herein encompass the administration of a remyelinating composition to a subject.

Such administration may encompass the administration of a pharmaceutically effective amount of the remyelinating composition, i.e. an amount sufficient to have a measurable effect on one or more remyelinating processes. For example, remyelinating composition dosages which result in physiological concentrations of 1 nM to 1 mM may be used, for example in the range of 5 nM to 500 nM. For estrogenic agents combined with remyelinating compositions, exemplary dosages include those which result in a physiological concentration in the range of 1 to 100 micromolar.

The subject of the administration may be any animal, for example, a human, a veterinary subject, or a test animal. In one embodiment, the subject is a human that is afflicted with a demyelinating condition or is at risk of having a demyelinating condition and is in need of treatment therefor. The scope of the invention also extends to the administration of remyelinating compositions to cells, cell cultures, and explanted tissues. In one embodiment, the scope of the invention encompasses the administration of a remyelinating composition to oligodendroglia in a micropillar assay, as known in the art.

In some embodiments, the administration of a remyelinating composition will be referred to as increasing, enhancing, or otherwise changing the magnitude of a myelination-associated process. Such change may be defined with respect to the magnitude of the myelination-associated process observed in untreated controls or in subjects prior to treatment.

In one aspect, the scope of the invention encompasses a method of treating a demyelinating condition in a subject by the administration of a remyelinating composition, wherein the remyelinating composition is a remyelinating SERM, a GPR56 agonist, or is both a remyelinating SERM and a GPR56 agonist. Alternatively, the scope of the invention may encompass the use of a remyelinating composition for the treatment of a demyelinating condition in a subject, wherein the remyelinating composition is a remyelinating SERM, a GPR56 agonist, or is both a remyelinating SERM and a GPR56 agonist. Treatment, as used herein, may include, for example; curing a demyelinating condition; ameliorating symptoms associated with demyelination (e.g. nerve signal disruption, axonal damage, and neurodegeneration); slowing the progression of a demyelinating condition; preventing or delaying the onset of a demyelinating condition in an at-risk subject; preventing further loss of myelin; or restoring myelin lost prior to treatment. Such treatment or use may be in combination with the administration one or more estrogenic agents.

In one aspect, the scope of the invention encompasses a method of enhancing remyelination of axons in a subject suffering from a demyelinating condition by the administration of a remyelinating composition to the subject. Enhancement, as used herein, refers to increasing the degree of remyelination, for example, increasing the thickness of the myelin sheath on axons, increasing the rate or prevalence of axon wrapping, increasing the rate of remyelination, or increasing the number of remyelinated axons in a treated area. Such administration may be in combination with one or more estrogenic agents.

In one aspect, the scope of the invention encompasses a method of enhancing the differentiation of OPCs into myelinating oligodendrocytes in a subject by the administration of a remyelinating composition to the subject. Enhanced differentiation, as used herein, may refer to in increase in any measure of OPC differentiation into myelin-producing oligodendrocyte cells, including, for example, increasing the proportion of differentiated oligodendrocytes to OPC's, for example, an increase in MBP expressing cells or a decrease in the proportion of PDGFRα expressing cells. Such administration may be in combination with one or more estrogenic agents.

In another aspect, the scope of the invention encompasses a method of using a remyelinating composition for the manufacture of a medicament for use in the treatment of a demyelinating condition. For example, a pharmaceutical composition for the treatment of a demyelinating condition may comprise one or more remyelinating compositions formulated or mixed in combination with one or more additional pharmaceutically acceptable elements. For example, remyelinating compositions may be combined with compositions such as carriers, excipients, diluents, buffers, salts, or release-modulating agents, as well as estrogenic compositions.

### EXAMPLES

### Example 1. Identification of Remyelinating SERMs

Various SERMs and other compounds were tested for their remyelination effects using the BIMA (Binary Indicant for myelination using Micropillar Arrays) assay, a functional high-throughput screen utilizing freestanding micropillar arrays of compressed silica around which myelin “rings” of membrane wrapping by oligodendroglia can be visualized in cross-section. BIMA allows testing of compounds' direct influences on oligodendroglia without indirect effects from neurons and other factors.

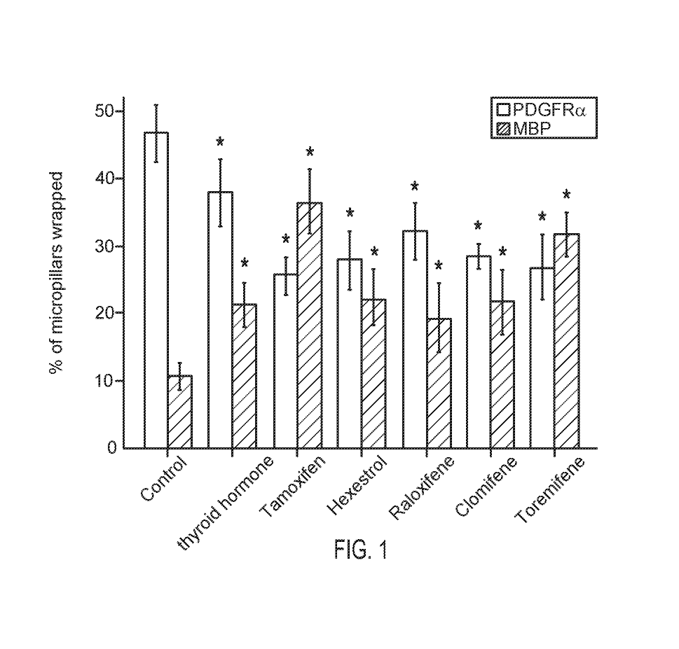

Micropillars were cultured with OPCs and the number of MBP-positive or PDGFRα-positive rings in each field of 100 micropillars was determined. Results are depicted in FIG. 1. Error bars represent mean±s.e.m.*P<0.05, significance based on Student's t-test with the respective controls. Seven additional SERMs or estrogen derivatives were screened at concentrations between 500 nM-1 μM without any significant effects on oligodendrocyte differentiation or membrane wrapping.

### Example 2. BZA Promotes Oligodendrocyte Differentiation

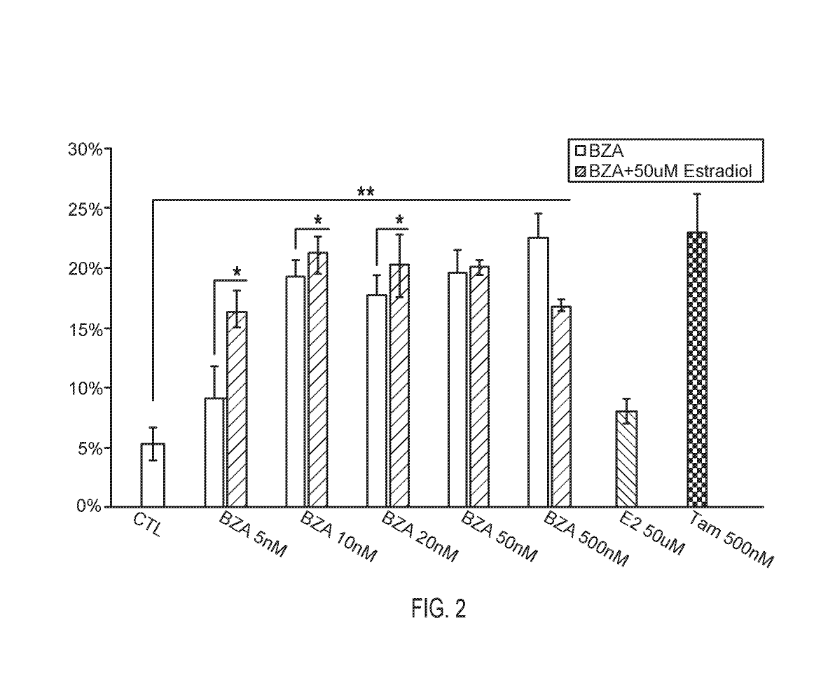

Purified OPCs were cultured and treated with different concentrations of BZA alone or with estradiol for 48 hours and immunostained for MBP, PDGFRα and DAPI. Quantification of the percentage of MBP-positive cells after treatment is depicted in FIG. 2. Error bars represent mean±s.e.m., *P<0.05, **P<0.01, significance based on Student's t-test with the respective controls.

### Example 3. OPC Differentiation

To assess the effect of selected SERMs on myelination, OPCs were derived from WT P7 rat pups using previously validated methodology, cultured in isolation, and treated with a select group of SERMs at a preliminary concentration of 500 nM for 48 hours. Cells were then permeabilized and immunostained for MBP (oligodendrocytes), PDGFRα (OPCs), and DAPI (cell bodies). Each SERM tested ((2,3-bis(4-hydroxyphenyl)-propionitrile, BZA and tamoxifen) significantly enhanced OPC differentiation (*p<0.05; **p<0.01; ***p<0.001) at this concentration. Additionally, OPCs were co-cultured with dorsal root ganglion neurons (DRGs) and subsequently treated with 500 nM BZA every 3 days. Co-cultures were fixed, permeabilized and stained. BZA significantly (p<0.001) enhanced OPC differentiation and subsequent myelination in the co-culture system, showing strong effects on OPC differentiation and myelination.

### Example 4. BZA Treatment on Human ESC-Derived OPCs

Human OPCs were generated from human ESCs and cultured for 10 days in the presence or absence of BZA (500 nM). The cells were then stained for 04 and MBP. There was a significant increase in the number of MBP-positive oligodendrocytes upon treatment with BZA.

### Example 5. Effects of BZA on OPC Differentiation

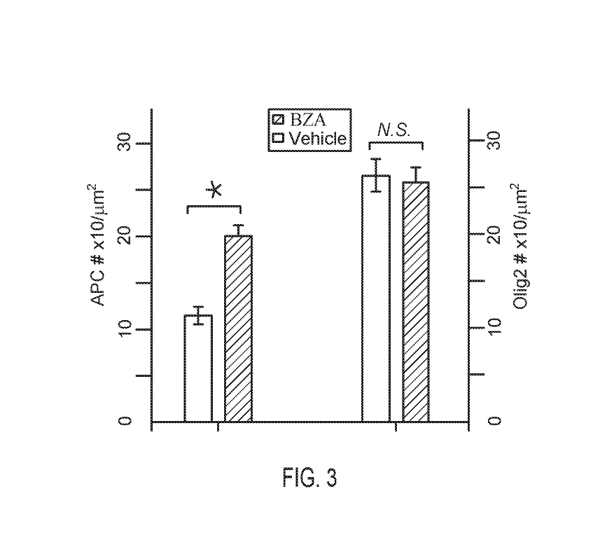

Lysolecithin was injected in the corpus callosum of mice and analyzed at 6 days after injection, showing a demyelinated lesion area. Mice were treated with either BZA (10 mg/kg) or vehicle control for 7 days after injection of lysolecithin. Mice were euthanized at 10 days post injection and brains were sectioned and immunostained for myelin oligodendrocyte glycoprotein peptide (MOG), Adenomatous polyposis coli (APC-a maker of mature oligodendrocytes) and Olig2. Quantification of APC-positive cells indicated a 2-fold increase in remyelination in the corpus callosum after treatment with BZA, as depicted in FIG. 3.

### Example 6. BZA Effects on Remyelination Kinetics

Toxic, focal demyelinating injury was induced in the corpus callosum of 8-week old adult mice the rate and extent of remyelination was assessed, using previously described methodology. Demyelination was induced by injecting 1 μl of 1% solution of lysolecithin. Essential to this demyelinating model is the timeline for repair, comprising active demyelination [1-3 days post lesion (dpi)], OPC recruitment (3-7 dpl, peaking at 5 dpl), oligodendrocyte differentiation (7-10 dpl) and active remyelination (14-21 dpl). Oligodendrocyte differentiation and remyelination at 10 dpl was analyzed, allowing assessment of BZA effects on the kinetics of myelin repair. BZA was administered via oral gavage to adult mice at a concentration of 10 mg/kg/day for 7 days following lysolecithin injections. Animals were sacrificed and perfused at 10 dpl. MOG and CC1/APC immunostaining demonstrated enhanced differentiation and significantly more oligodendrocytes in the lesion of BZA-treated mice at 10 dpl when compared to littermate vehicle-treated controls. These findings further demonstrate that BZA greatly promotes differentiation and accelerates the kinetics of remyelination after a demyelinating insult.

### Example 7. Remyelination Effects are Independent of Estrogen Receptor Activity

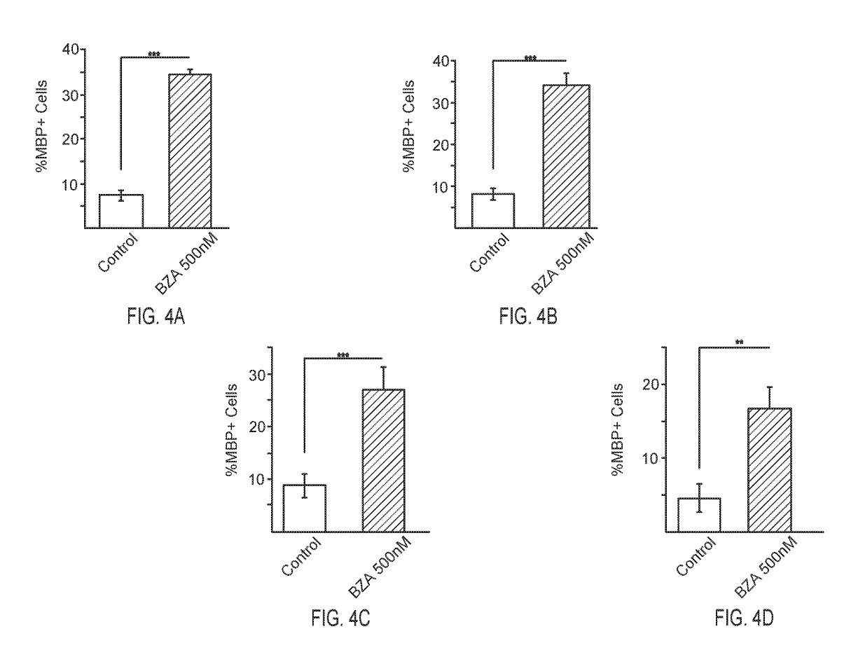

OPCs were isolated from estrogen receptor alpha (ERα) or beta (ERβ) null P7 mice, as well as from double knockout animals. Knockout animals were confirmed via genotyping for the inserted cassette into either the Esr1 or Esr2 gene (genes that encode for ERα and ERβ, respectively). OPCs were cultured in isolation, and treated with 500 nM BZA for 48 hours. Cells were then permeabilized and stained for MBP (oligodendrocytes), PGFRα (OPCs), and DAPI (cell bodies) MBP/DAPI+ cells/total cells were quantified under 20× magnification. Significance was determined used two-tailed Student's t-test (*p<0.05; **p<0.01; ***p<0.001). BZA significantly enhanced OPC differentiation in wild type (FIG. 4A), ERα null (FIG. 4B), ERβ null (FIG. 4C), and ERα/ERβ null mice (FIG. 4D), when compared with control. These results demonstrate that ERs are not necessary for SERM remyelinating agents to elicit remyelination effects.

### Example 8. Remyelinating SERMs Activate GPR56

As previous findings demonstrated that SERMs do not act through the estrogen receptors, a bioinformatics screen was conducted to identify the molecular target of the remyelinating SERMs which promotes remyelination, employing a bioinformatics approach which integrates chemical and molecular profiling. Specifically, the analysis was designed to identify non-overlapping targets between estrogen derivatives (which alone have no effects on myelination) and the remyelinating SERMs. The top candidate identified was GPR56, an adhesion GPCR which has previously been implicated as playing a role in oligodendrocyte development. Preliminary data suggests that GPR56 is the molecular target of the remyelinating SERMs for promotion of remyelinating effects.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

What is claimed is:

1-25. (canceled)

26. A method of promoting remyelination in demyelinated tissue of a subject, comprising
administering to the subject a remyelinating SERM.

27. The method of claim 26, wherein
the remyelinating SERM is selected from the group consisting of bazedoxifene, raloxifene, clomifene, ospemifene, lasofoxifene, diarylpropionitrile, toremifene, and tamoxifen.

28. The method of claim 27, wherein
the remyelinating SERM is bazedoxifene.

29. The method of claim 26, further comprising
the co-administration of an estrogenic agent.

30. The method of claim 29, wherein
the estrogenic agent is selected from the group consisting of estrogen, estradiol, conjugated estrogens, estriol, estrogen mimic, estrone, ethynil estrogen, and estrogen agonist.

31. The method of claim 26, wherein
the subject has a demyelinating condition of the central nervous system.

32. The method of claim 31, wherein
the demyelinating condition of the central nervous system is selected from the group consisting of a myelinoclastic disorder, MS, Devic's disease, an inflammatory demyelinating disease, an acute disseminated encephalomyelitis, a leukodystrophic disorder, central nervous system neuropathy, central pontine myelinolysis, and progressive multifocal leukoencephalopathy.

33. The method of claim 31, wherein
the demyelinating condition is MS.

34. The method of claim 26, wherein
the subject has a demyelinating condition of the peripheral nervous system.

35. A method of promoting oligodendrocyte precursor cells in a subject to differentiate into myelin-producing oligodendrocytes, comprising
administering to the subject a remyelinating SERM.

36. The method of claim 35, wherein
the remyelinating SERM is selected from the group consisting of bazedoxifene, raloxifene, clomifene, ospemifene, lasofoxifene, diarylpropionitrile, toremifene, and tamoxifen.

37. The method of claim 36, wherein
the remyelinating SERM is bazedoxifene.

38. The method of claim 35, further comprising
the co-administration of an estrogenic agent.

39. The method of claim 38, wherein
the estrogenic agent is selected from the group consisting of estrogen, estradiol, conjugated estrogens, estriol, estrogen mimic, estrone, ethynil estrogen, and estrogen agonist.

40. The method of claim 35, wherein
the subject has a demyelinating condition of the central nervous system.

41. The method of claim 40, wherein
the demyelinating condition of the central nervous system is selected from the group consisting of a myelinoclastic disorder, MS, Devic's disease, an inflammatory demyelinating disease, an acute disseminated encephalomyelitis, a leukodystrophic disorder, central nervous system neuropathy, central pontine myelinolysis, and progressive multifocal leukoencephalopathy.

42. The method of claim 41, wherein
the demyelinating condition is MS.

43. The method of claim 36, wherein
the subject has a demyelinating condition of the peripheral nervous system.
